(19) United States
(12) Patent Application Publication
(10) Pub. No.: US 2011/0106190 A1
(43) Pub. Date: May 5, 2011

(54) Defibrillator Having a Secure Discharging Circuit Comprising an H-Bridge

(75) Inventors: Clement Foeller, Lampertheim (FR); Alfred Schiller, Aeugst a.A. (CH); Albert Cansell, Wissembourg (FR)

(73) Assignee: SCHILLER MEDICAL S.A.S., Wissembourg Cedex (FR)

(21) Appl. No.: 11/722,677

(22) PCT Filed: Dec. 21, 2005
(86) PCT No.: PCT/EP2005/056993

§ 371 Date: Dec. 23, 2010

(30) Foreign Application Priority Data
Dec. 23, 2004 (FR) 413869

(51) Int. Cl. A61N 1/39

(52) U.S. Cl. 607/5

(57) ABSTRACT

The invention relates to a cardiac defibrillator used to treat a patient in cardio-circulatory arrest by a shock from a dosed biphasic discharge from a capacitor through an H-bridge comprising a high-voltage switch A, B, C or D in each of the limbs thereof. Said cardiac defibrillator is characterised in that each opposing polarity phase of the biphasic shock is controlled in two stages in such a way that, for each pair of switches associated with a phase, the first switch is switched on and remains on during the entire phase, while the second switch switches off in a staggered manner in relation to the first switch for a controlled amount of time in order to pass the current through the patient during said phase, the same process being carried out for the second phase with the other pair of switches. The invention is especially suitable for manufacturers of defibrillation appliances.

The present invention relates to the medical field and more particularly to emergency cardiac resuscitation in the event of cardio-circulatory arrest following ventricular fibrillation or ventricular tachycardia, and its subject is an external cardiac defibrillator.

Emergency cardiac defibrillation has experienced a considerable upsurge and considerable development in recent years.

Cardiac defibrillation is the only means of reducing heart attacks due to fibrillation or to ventricular tachycardia which irretrievably lead to death if they are not treated by a defibrillation shock within the space of a few minutes.

Originally, up to about 10 years ago, the use of a defibrillator was limited to emergency doctors only, who alone were authorized to use such apparatus and who alone were provided therewith.

This situation being quite inadequate given the low chance of it being possible for an emergency doctor to be at the site of the incident within a short enough time to save the subject, there have been moves, as a first stage, for defibrillators to be used by professional first-aiders such as professional firemen, since there are more of them and they provide much wider cover than emergency doctors. The apparatus widely used at present by such personnel are of the Semi-Automatic Defibrillator (SAD) type. The principle of this type of apparatus consists in the apparatus automatically detecting a disturbance in the rhythm requiring defibrillation and recommends the first-aider to apply a shock.

As a second stage, these semi-automatic defibrillators have begun to be extended to a much wider population of users, including the general public: these SADs are then commonly

called PADs (“Public Access Defibrillators”), that is to say defibrillators that can be used by members of the public who have received minimal training in first-aid.

These latter types of apparatus: SAD or PAD naturally always presuppose the presence of a third party who is present precisely in proximity to the victim of the cardio-circulatory arrest and to whom such an apparatus is available.

Since this condition is not acceptable in the case of patients who are known to be subject to fibrillation attacks that could occur at any time, the implantation of an automatic implantable defibrillator which applies the shock if necessary has been envisaged. However, the implantation of such an apparatus being cumbersome and invasive for the patient, an alternative has been developed for such patients subject to recurring fibrillations, if appropriate waiting for the implantation of an implantable defibrillator, which consists of an automatic external apparatus worn by the patient.

Such an apparatus is for example described in document EP 1 064 963: the apparatus worn by the patient continuously monitors the subject's rhythm, and in the event of ventricular fibrillation automatically triggers a defibrillation shock by way of electrodes applied to the chest.

The field of application of the present patent relates to these various types of defibrillators, whether they are external and used by third party doctors or first-aiders, inside or outside a hospital, or whether they are external and worn by the patient, or whether they are implantable, as well as defibrillators with a stimulation function that are frequently placed in the general category of defibrillators and that are dubbed solely thus.

The invention consists of a cardiac defibrillator intended to treat a patient with cardio-circulatory arrest following fibrillation or ventricular tachycardia by means of at least one

biphasic defibrillation shock consisting of a wave with at least two phases of opposite polarities, each obtained by means of an H bridge comprising two pairs of high-voltage switches, characterized in that each of the opposite phases of the biphasic wave is controlled in two stages such that, for each pair of high-voltage switches respectively relevant to a given phase, one of the switches of this pair is rendered conducting in a first stage and remains conducting throughout the phase and that the second high-voltage switch of this pair which is in series in the circuit incorporating the patient, is turned on in a second stage so as to establish the current through the patient during this phase.

The H bridge comprises four switches A, B, C, D, the shock being applicable to a load outside the apparatus through the H bridge. The two switches A and B are each linked on one side to the high-voltage capacitor CHT at the point Z and are each linked on the other side respectively to a point X and Y intended to be connected to the load outside the apparatus. The other two switches C and D are each connected on one side respectively to the point X and Y intended to be connected to the outside load and on the other side to a point W, in particular earthed, having a lower potential than the point Z. The pairs of switches A+D and B+C are used respectively for the first and the second phase of each defibrillation pulse. A control circuit controls one of the switches A or B for each phase so as to switch it on individually during the corresponding phase of the biphasic wave. A control circuit controls the switches C and D, through which they are switched from the initial off state to the on state during each of the successive phases of the biphasic wave but only after the corresponding switch A or B is turned on.

The invention will be better understood by virtue of the description below, which pertains to preferred embodiments, given by way of nonlimiting examples, and explained with reference to the appended schematic drawings, in which,

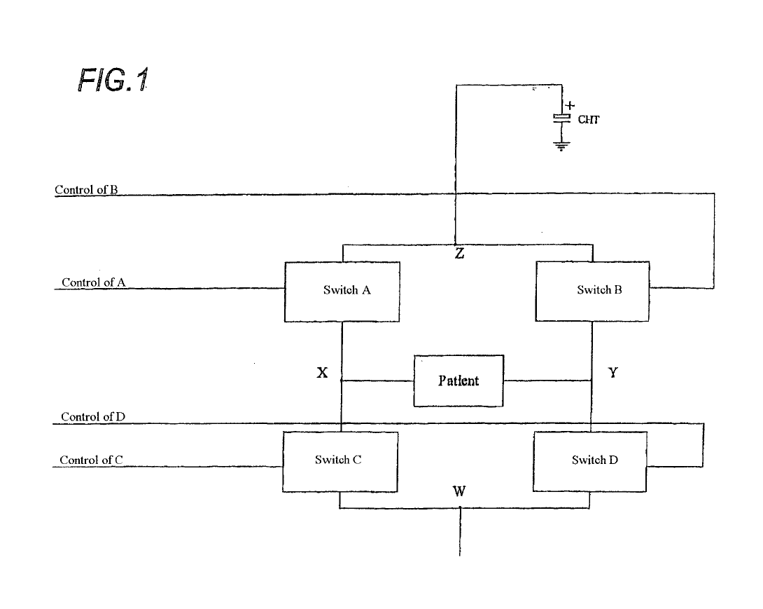

FIG. 1 is a basic diagram of the H bridge intended to generate a biphasic defibrillation pulse through a patient of the defibrillator according to the invention,

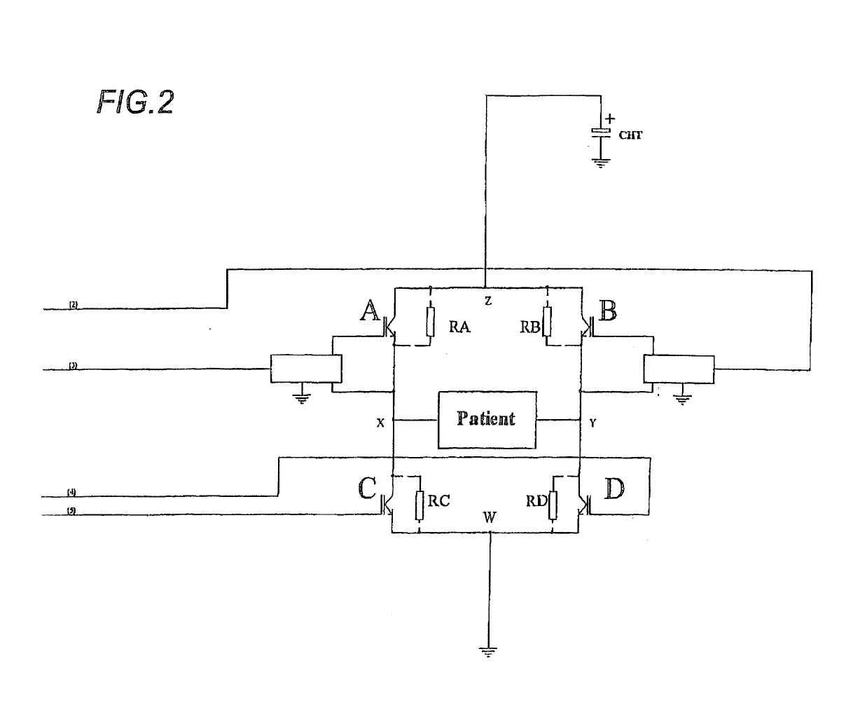

FIG. 2 is a more detailed electrical diagram of the circuit using an H bridge intended to generate a biphasic defibrillation pulse through a patient, of the defibrillator according to the invention,

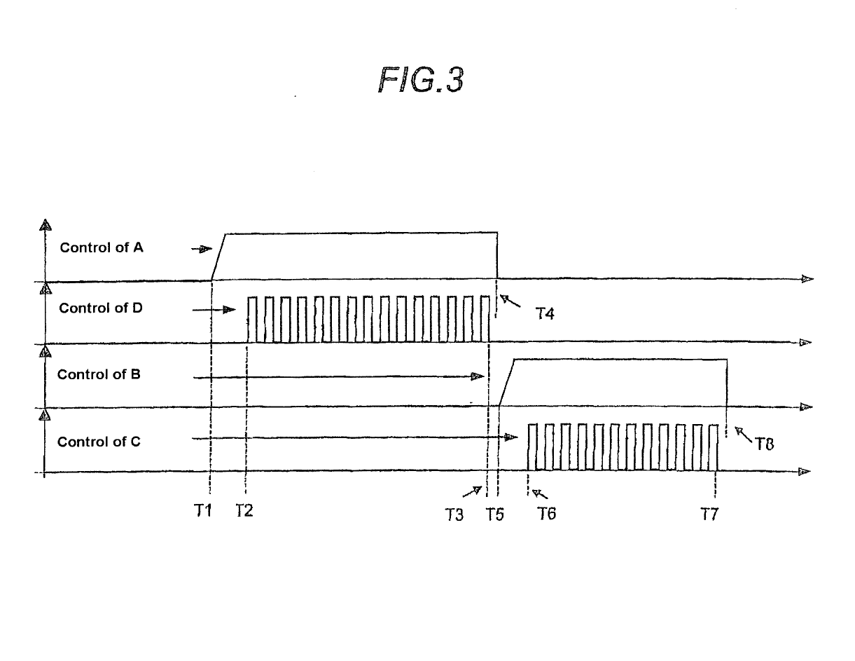

FIG. 3 is a timing diagram of the control of the four switches of the H bridge in the particular case where the two phases to be obtained are sliced or chopped,

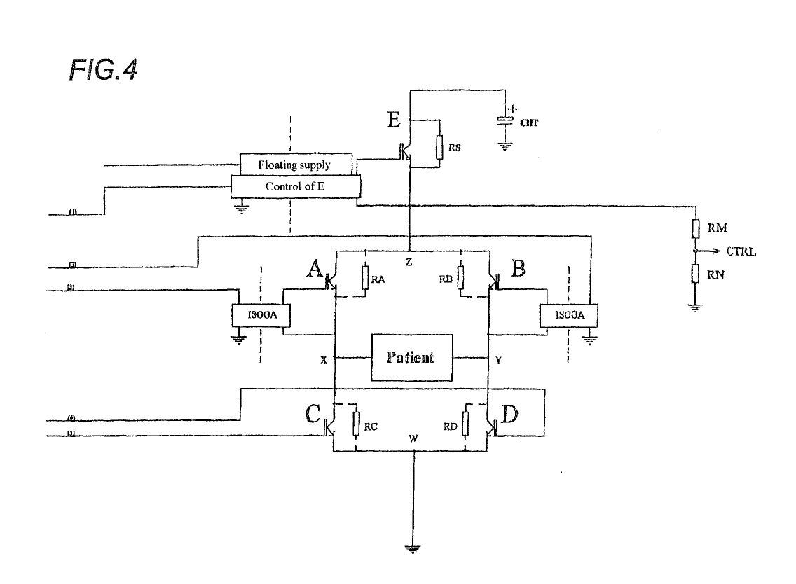

FIG. 4 is a diagram of an exemplary embodiment which comprises a fifth switch, consisting of an IGBT, the aim of which is to cut off the high voltage arriving at the H bridge, before and after the shock,

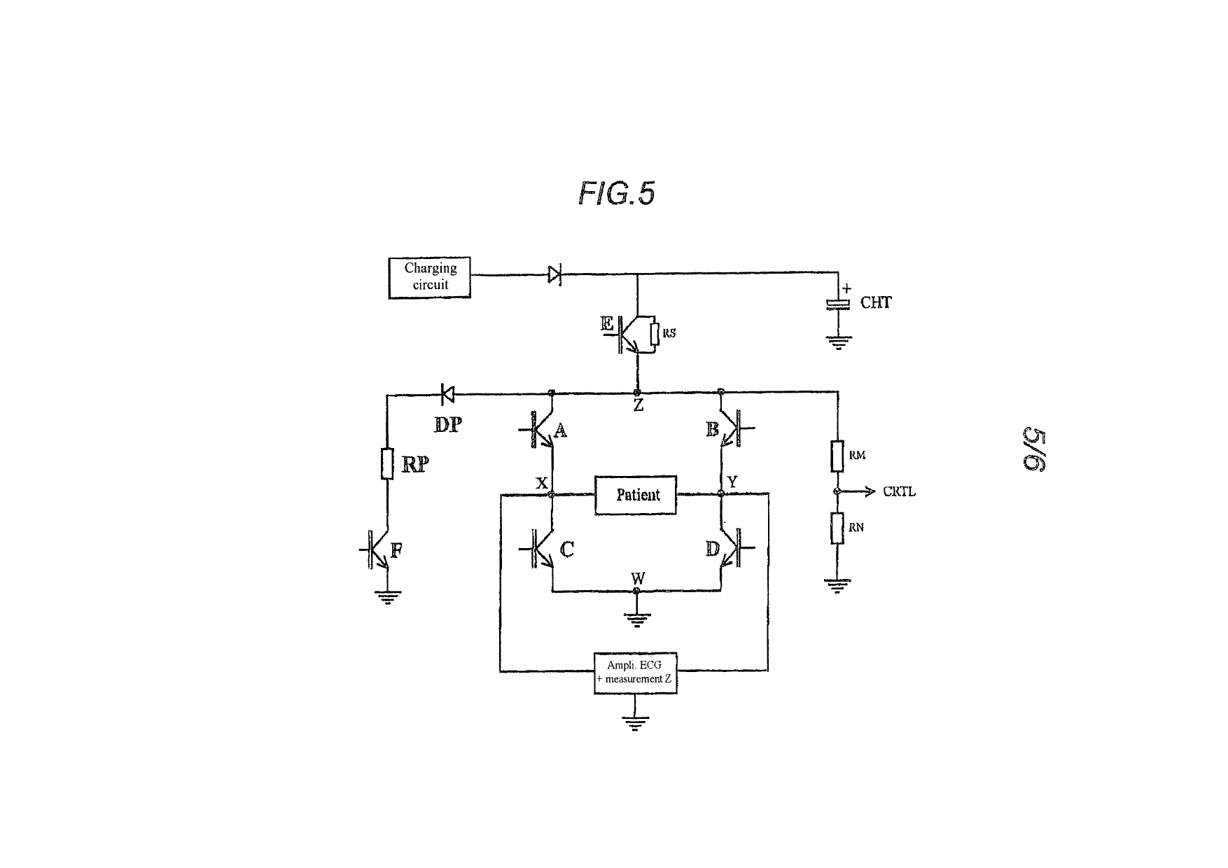

FIG. 5 is a simplified diagram limited to the central part of the circuit without any balancing resistors and exhibiting a branch for reducing the electrical glitches stemming from the charging of the high-voltage capacitor as well as a divider bridge allowing the monitoring of the IGBTs,

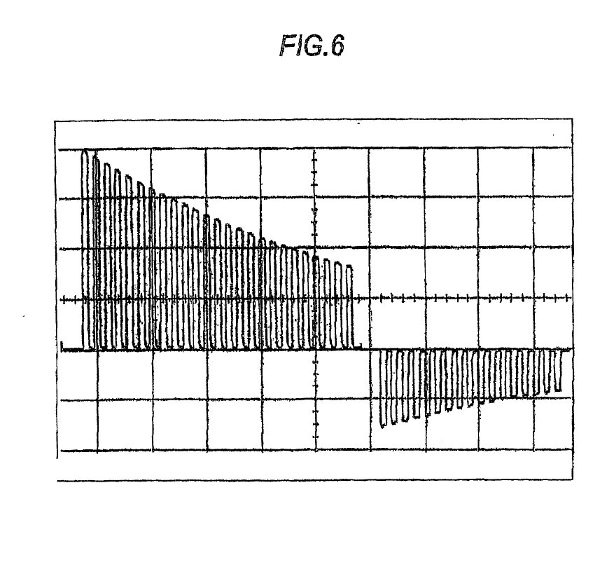

FIG. 6 is a time-chart representation of the image of the current passing through the patient during a defibrillation shock with chopped pulses.

The basic schematic diagram of the invention is illustrated by FIG. 1. This figure shows a high-voltage capacitor CHT which feeds an H bridge consisting of four switches A, B, C and D that can be controlled by four respective control lines. The high voltage coming from the capacitor CHT is applied to the upper point Z of the H bridge, with respect to the earth connected to the point W at the bottom of the H bridge. The intermediate point between the switches A and C is called X, the intermediate point between the switches B and D is called Y. X

and Y constitute the diagonal of the H bridge which goes to the patient. In the more detailed electrical diagram of FIG. 2, given by way of example, the four switches of the H bridge, namely A, B, C and D, consist of four high-voltage semiconductor switching components controlled or triggered by a signal, for example insulated gate bipolar transistors known in the art by the term IGBT that will be used for the remainder of the description.

High-voltage resistors of large value RA, RB, RC and RD (for example 40 Mohms) are for example wired in parallel between the collector and the emitter of each IGBT, respectively A, B, C, and D, so as to have well defined potentials between the IGBTs in the off state. This allows, on the one hand more reliable and more secure operation, and on the other hand, makes it possible, by measuring the voltages appearing at the points of the junctions, to detect any defects in the IGBTs, in particular any short-circuit.

These resistors are represented diagrammatically not connected in FIG. 4 since they turn out to be optional.

The use of the leakage resistance (internal resistance in the disabled state) individual to each IGBT transistor as replacement for the resistors RA, RB, RC, RD used for balancing the bridge has been envisaged. The operating principle remains the same. It suffices to allow for the spread in the values of the leakage resistances of the IGBTs during measurements.

This variant is represented in FIG. 5. However, this leakage resistance is difficult for semiconductor manufacturers to control, and may vary as a function of temperature and of the voltage applied to the transistor.

For this reason, an external divider bridge RM-RN which constitutes another advantageous solution making it possible to detect a defect in the IGBTs has been provided in the arrangement of FIGS. 4 and 5 as replacement for the resistors RA, RB, RC and RD.

The process according to the invention for delivering a bi-phasic shock is as follows with reference to FIG. 1. A command arriving at the control of the switch A turns the latter on. After a time interval, for example around 0.5 ms, the command for the switch D arrives, and the latter in turn becomes conducting. The current from the high-voltage capacitor CHT is established through the patient through the switches A and D to earth for the instructed duration, for example around 4 ms, this constituting the first phase of the shock. Once the current has been cut by A and D, the second phase begins because the switch B is rendered conducting by a corresponding command arriving at its input. In a similar manner to the first phase, the switch C is controlled with a delay with respect to B; i.e. for example around 0.5 ms after B is turned on, the command to turn on the switch C arrives, and the latter in its turn becomes conducting. The current from the capacitor CHT is then established again through the patient via the switches B and C to earth for the instructed duration, i.e. around 4 ms, this constituting the second phase of the biphasic shock.

All types of controls and modulation of control of the switches D and C are possible from total and continuous conduction up to control by chopping with variation in the shape factor which makes it possible to dose the energy applied according to a predetermined law or with pulse modulation or any other form of modulation.

A preferred mode of this process consists in slicing or chopping the two phases at a higher frequency than the frequency of said successive phases, a frequency of 5 kHz for example. The process is the same as that just described, except that the controls for turning on the switches D (for the first phase) and C (for the second phase) are not continuous, that is to say are not applied during these phases for example at a permanent high level as in the example described above, but receive a signal which is chopped or sliced, or even modulated between the high level and 0 volts. This mode of operation, similar to the previous one but more general, is illustrated by FIG. 3 which shows the timing diagram for the control signals of the four switches:

T1 corresponds to the turning on of A

T2 corresponds to the turning on of D in a chopped manner

T3 corresponds to the turning off of D

T4 corresponds to the turning off of A

T5 corresponds to the turning on of B

T6 corresponds to the turning on of C in a chopped manner

T7 corresponds to the turning off of C

T8 corresponds to the turning off of B.

As may be seen in the shape of the curves of FIG. 6 obtained on the basis of control signals similar to those described and represented in FIG. 3, the shock thus delivered to the patient is a sliced or chopped biphasic pulse.

If the commands for turning on C and D were not chopped but continuous, the biphasic pulse obtained would include a positive phase and a negative phase with continuous decay, this corresponding to the conventional biphasic pulse with continuous-decay truncated exponentials for each of the phases.

This mode of switching by two-staged turning on of the switches such as insulated gate transistors IGBT (FIG. 2) for each of the two phases, affords excellent reliability.

A transistor used in switching operates principally in two states, i.e. off, or on. Toggling from the off state to the on state is effected through a transition which should usually be as short as possible so as to avoid damaging the transistor.

Specifically, in the off state, no current (except for leakage currents) passes through the transistor but the voltage across its terminals (points Z and X for transistor A or Z and Y for transistor B) is a maximum. In the on state, the current which passes through the transistor is a maximum, but the voltage across its terminals is almost zero. The power and hence the energy dissipated by the transistor is then low both in the off state and in the on state.

During the switching phase (toggling from the off state to the on state or vice versa), the transistor passes through a transient period in the course of which the current increases gradually from zero up to the maximum while the voltage passes from the maximum to a near-zero value. Stated otherwise, the transistor passes through a phase where the power and hence the energy dissipated may be very significant. If this transient phase lasts too long, the transistor may be destroyed on account of excessive heating.

To guarantee proper operation, and optimal reliability and longevity of the transistor, it is therefore necessary to limit the power and hence the energy dissipated by it.

This limitation may be obtained in various ways.

The first consists in minimizing the duration of this transition phase. The second consists in making the transistor switch in the absence of current. In the latter case, the switching duration is no longer as critical.

The use of a galvanically isolated control to control the transistors A and B, insofar as it must remain simple so as to minimize the number of components and reduce the electrical consumption of the circuit, does not usually make it possible to obtain fast switching of the transistors A or B.

The turning on of the transistors A or B before the passage of the current, which flows only when D or C is on, therefore makes it possible to avoid the dangerous dissipation of energy in the transistors A and B and hence ensures reliable operation.

This mode of switching and of layout makes it possible moreover not to be compelled to isolate at high voltage the control of the IGBTs C and D. They are controlled with respect to earth, thereby allowing them to be switched easily either continuously so as to obtain two phases consisting of conventional continuous truncated exponentials as in the first variant of the invention, or as two phases chopped according to any chopping law, shape factor or pulse modulation as in the second variant of the invention or any other form of modulation.

The control of C and D with respect to earth also allows the use of a simple control circuit affording fast switching which ensures a minimum of dissipation and excellent reliability for these transistors which switch a high current, unlike A and B.

A particular consideration in respect of this kind of IGBT-based defibrillation circuit relates to the safety of the patient.

Specifically, should one of the IGBTs be destroyed, a current might reach the patient before the shock is applied. This current would be dangerous.

The state of the art for ensuring sufficient safety with respect to the patient when using semiconductor circuits to generate a defibrillation shock through a patient is given for example by document U.S. Pat. No. 5,824,017. In this document, which also describes the use of a semiconductor H bridge, it is seen that the patient is separated from the H bridge by an electromechanical relay with two contacts. The contacts of this relay are permanently open and close only at the precise moment at which the shock should be given. In this way, it is guaranteed that no dangerous current can reach the patient other than at the time at which the shock is applied.

However, since such an electromechanical relay is relatively bulky and consumes an appreciable current, the inventors have attempted to devise safety devices that are secure enough to be able to avoid the use of less reliable electromechanical relays than the solution adopted.

The particularly advantageous safety devices thus provided for within the framework of this invention are as follows:

A fifth IGBT referenced E is provided in series between the high-voltage capacitor CHT and the H bridge (FIG. 4). This fifth IGBT referenced E is permanently off while the shock is not given, and is on only during the shock. In this way, the H bridge is totally cut off from the capacitor before the shock, thereby avoiding any risk of current through the patient before or after the shock. This IGBT referenced E is also furnished with a parallel resistor RS of high value (for example 40 Mohms between the collector and the emitter, so as to pass a weak current for verifying the proper operation of the H bridge.

The fifth IGBT referenced E is also controlled by a circuit arriving at the gate of E through a galvanic isolation arrangement, this arrangement being fed with a floating supply as represented in FIG. 4.

In order to permanently monitor whether the IGBTs of the H bridge are in good condition before the application of the shock and to detect any defect in one of them such as for example a short circuit, a safety circuit provided by the invention consists in measuring at any time the voltage at the point Z between the IGBT referenced E and the H bridge. This voltage must have a value lying within well defined limits. It depends on the resistances of the branches of the bridge in the nonconducting state and is measured with the aid of the divider bridge represented by the resistors RM and RN in the right-hand part of FIG. 5 which between them define a measurement output dubbed CTRL in FIGS. 4 and 5. It also depends on the values of the resistors RA, RB, RC and RD when they are for example chosen to be equal and high (for example 40 Mohms) placed in parallel with each of the five IGBTs. If for any reason one of the IGBTs was short-circuited, then it would have to be turned off, this voltage would drop in a consequent manner, and this would be detected by the system and would shut down the operation of the apparatus and prevent its use so as to eliminate any risk to the patient.

Another method that may be used alternatively or in addition consists (considering the example of FIG. 2) in measuring and monitoring, permanently, in the absence of the shock, the potential difference between the points of the diagonal X and Y of the H bridge. Normally this potential difference is practically zero, given the symmetry of the circuit and the possible presence of the resistors of high equal values, arranged in parallel with the IGBTs. If on the other hand one of the IGBTs had for example to be short-circuited, the bridge would be greatly unbalanced, and this would give rise to a large voltage difference between X and Y. This measurement may be made either by a differential measurement directly between the points X and Y, or by inserting between the resistors of high value (for example 40 Mohms) RC and RD and earth, resistors of lower value (for example 10 Kohms) and thus creating two voltage dividers whose outputs with respect to earth will be indicative, should a large voltage appear, of a fault with an IGBT.

An embodiment which is advantageous as regards the IGBTs having to be isolated from earth (A, B and E) consists in their being controlled through a galvanic isolation arrangement ISOGA according to various means, for example optoelectronic with photoelectric coupler and photovoltaic, with high-frequency transformer controlled by high-frequency pulses or any other appropriate isolation arrangement. Each of them is represented by a rectangle referenced ISOGA.

Another variant of the circuit is represented in FIG. 5. It exhibits an additional branch for reducing electrical disturbances and glitches stemming from the charging of the high-voltage capacitor CHT. This branch extends from the point Z to earth. It comprises a diode DP, a resistor RP and an insulated gate transistor F for example of the IGBT type which is rendered conducting during the charging of the capacitor CHT. The voltage divider bridge formed by the resistor RS and this earthed branch makes it possible by virtue of the value of RP (for example 5 Kohms) to appreciably reduce the amplitude of the electrical glitches at the point Z stemming from the charging of the capacitor CHT through a voltage multiplier represented by the charging circuit of FIG. 5. The glitches arriving at the H bridge are thus sufficiently weak.

This branch RP+DL exhibits an additional function. It makes it possible, for safety reasons, by simultaneously rendering the transistors E and F conducting, to discharge the capacitor CHT.

The role of the diode DP consists in maintaining the line Z at a low but non-zero potential so as to decrease the leakage currents in the IGBTs while allowing correct operation of the amplifier ECG and measurement of the impedance of the patient as indicated by ampli. ECG and measurement Z in FIG. 5.

This makes it possible to guarantee lower values for any leakages to the patient.

1. A cardiac defibrillator intended to treat a patient with cardio-circulatory arrest following fibrillation or ventricular tachycardia by means of at least one defibrillation shock consisting of a defibrillation pulse forming a biphasic wave having at least one first phase and one second phase of opposite polarities, the defibrillator comprising
a high-voltage capacitor for generating a shock, the shock being obtained by discharging a high-voltage capacitor from a point Z and
an H bridge comprising four switches A, B, C, D, the shock being applicable to a load outside the apparatus through the H bridge,
the two switches A and B each being linked on one side to the high-voltage capacitor at the point Z and each being linked on the other side respectively to a point X and Y intended to be connected to the load outside the apparatus,
the other two switches C and D each being connected on one side respectively to the point X and Y intended to be connected to the outside load and on the other side to a point W, in particular earthed, having a lower potential than the point
and the pairs of switches A+D and B+C being used respectively for the first and the second phase of each defibrillation pulse,
characterized by a control circuit which controls one of the switches A or B for each phase so as to switch it on individually during the corresponding phase of the biphasic wave,
and by a control circuit, which controls the switches C and D, and through which they are switched from the initial off state
to the on state during each of the successive phases of the biphasic wave but only after the corresponding switch A or B is turned on.

2. The defibrillator as claimed in claim 1, wherein the switches A and B linked to the high-voltage capacitor CHT remain on throughout the duration of the respective phases.

3. The defibrillator as claimed in claim 1, wherein the switches D and C remain on respectively during said first and second phases, thereby creating the generation of a defibrillation pulse of conventional biphasic truncated exponential type.

4. The cardiac defibrillator as claimed in claim 1, wherein the second switch of each pair which is intended to be connected in series with the load outside the apparatus after having remained off for a given duration at the start of the respective phase is turned on with respect to the point W, in particular to earth, so as to be turned on and off successively throughout the remainder of this same phase so as to establish a sliced or chopped current through this outside load.

5. The cardiac defibrillator as claimed in claim 4, wherein the two successive phases of opposite polarities are sliced or chopped at a higher frequency than the frequency of said successive phases.

6. The defibrillator as claimed in claim 4 wherein the switches D and C are controlled respectively for the first and second phases by a sliced or chopped signal, while the switches A and B are respectively turned on for the respective phases, thereby creating the generation of a defibrillation pulse of sliced or chopped type consisting for each phase of a train of pulses separated by pauses and exhibiting any shape factor or any pulse modulation.

7. The defibrillator as claimed in claim 1, wherein a fifth, safety switch E is interposed in the connection coming from the high-voltage capacitor so as to cut off any voltage arriving at the H bridge before and after the shock.

8. The defibrillator as claimed in claim 1, wherein the five switches are TGBTs each having a resistance of large value between their respective collector and respective emitter.

9. The defibrillator as claimed in claim 7, further comprising means for measuring or monitoring the voltage present at the level of the safety switch E at the point Z which is the top of the H bridge during the charging of the capacitor and before the delivery of the shock, so as to detect whether this voltage drops below a certain value, which would be indicative of the presence of a possible defective component among the switches of the H bridge.

10. The defibrillator as claimed in claim 9, further comprising detection means for detecting the possible voltage drop at Z by measuring the voltage by a divider bridge, that is to say between two resistors in series which connect the point Z to earth.

11. The defibrillator as claimed in claim 1, wherein each of the three switches A, B and E wired to the high voltage is controlled on its insulated gate by means of a galvanic isolation arrangement.

12. The defibrillator as claimed in claim 11, wherein the galvanic isolation arrangement is an optocoupler system ensuring isolation.

13. The defibrillator as claimed in claim 11, wherein the galvanic isolation arrangement is a high-frequency transformer system ensuring isolation.

14. The defibrillator as claimed in claim 1, further comprising, between the point Z and the point W, a branch which is composed in series of a diode DP, of a resistor RP and of a transistor F with insulated gate for example of the IGBT type which is rendered conducting during the charging of the capacitor and in that the voltage divider bridge using this branch between the point Z and the point W makes it possible by virtue of the value of the resistors, that at the terminals of E and RP, to appreciably reduce the amplitude of the electrical glitches at the point Z stemming from the charging of the capacitor through a voltage multiplier and in that this branch makes it possible, by rendering the transistors E and F conducting, to discharge the capacitor of its electrical energy.

15. The defibrillator as claimed in claim 14, further comprising a diode DP which is intended to maintain a low potential at Z.

16. A method of operating a defibrillator, as claimed in claim 1, so as to generate a biphasic defibrillation wave comprising two phases of opposite polarity by means of a capacitor and an H bridge comprising four high-voltage switches A, B, C, D, one switch in each of its vertical branches wherein
each of the biphasic defibrillation phases is controlled in two stages by rendering one of the switches conducting during a given phase for each pair of switches A-D and B-C, the other switch of this pair which is in series in the circuit incorporating a load outside the apparatus being turned on after a delay so as to be controlled in the desired manner throughout the relevant phase.

17. The method as claimed in claim 16, wherein the control of the other switch is a chopping control according to a certain shape factor.

18. The method as claimed in claim 16, wherein the control of the other switch is a pulse modulation control.
